(19) United States
(12) Patent Application Publication
(10) Pub. No.: US 2012/0115232 A1
(43) Pub. Date: May 10, 2012

(54) METHOD FOR INDUCING DEGRADATION OF PROTEIN IN MAMMALIAN CELL

(75) Inventors: Masato Kanemaki, Osaka (JP); Tatsuo Kakimoto, Osaka (JP); Kohei Nishimura, Osaka (JP); Haruhiko Takisawa, Osaka (JP); Tatsuo Fukagawa, Shizuoka (JP)

(73) Assignee: OSAKA UNIVERSITY, Suita-shi (JP)

(21) Appl. No.: 13/266,695

(22) PCT Filed: Nov. 5, 2009
(86) PCT No.: PCT/JP09/05863

§ 371 Date: Jan. 23, 2012

(30) Foreign Application Priority Data
Apr. 30, 2009 (JP) 2009-110449

(51) Int. Cl. C12N 5/10; C12N 15/85

(52) U.S. Cl. 435/455; 435/325; 435/320.1; 435/375

(57) ABSTRACT

Provided is a system which can induce the degradation of a protein of interest in a mammalian cell system reliably and stably within a short time. A mammalian cell inducible for protein degradation, the degradation of a protein of interest being induced by an auxin, in which the mammalian cell has both a TIR1 family protein gene from rice and a chimeric gene expressing a protein of interest labeled with a plant Aux/IAA family protein.

### TECHNICAL FIELD

The present invention relates to a method for efficiently inducing degradation of a protein of interest in mammalian cells.

### BACKGROUND ART

Controlling the expression level of a specific protein in a cell is extremely useful for the analysis of the function of the protein and the biological phenomena in which the protein is involved in the cell. For this purpose, various methods, each of which is based on a separate principle, have been developed to date.

For example, a method of tetracycline-dependent transcription and expression of a gene transferred into culture cells which utilizes a tetracycline-binding transcriptional repressor of E. coli is known (Non Patent Documents 1 and 2). The transcriptional repression system based on this principle is commercially available, and many researchers have actually used it to achieve successful results. However, given that this method aims at the transcriptional regulation of a gene of interest, in many cases even once inhibition of the expression is carried out, it takes a long time until the actual protein is depleted from inside the cell. Also, under certain circumstances, a partial reduction of the expression level of a protein of interest may inadvertently activate secondary intracellular responsive reactions.

Similarly, as a method of inhibiting the expression in culture cells, a method of transferring siRNA and shRNA into the cells to degrade mRNA, which is the product of a gene of interest, utilizing RNA interference reaction in the cells is known. In fact, products based on this method are also sold by many companies. However, given that this method also inhibits expression at the mRNA level, it generally takes as long as 24 to 48 hours for inhibition of the expression to take place. Also, the degree of inhibition of the expression depends on the target factor and the target RNA sequence, and thus the chances that inhibition of the expression is accomplished by 90% or more are not so high.

Recently, a method for regulating protein expression, including introducing a degradation-inducing tag to the target protein and regulating the degradation of the tag, has just been put into practical use. A protein obtained through modification of rapamycin-binding protein FKBP12 is stabilized upon binding to Shield 1, which is a rapamycin-analog compound, whereas if it is left unbound, it becomes unstable and is degraded within the cells. Utilizing this property, a method of Shield 1-dependent regulation of the expression, including fusing this FKBP12-derived protein to a protein of interest, was published (Non Patent Document 3). This method is generally assumed to degrade the target protein approximately in four hours through elimination of Shield 1, and a set of necessary plasmids and the like are on the market. This method requires constant addition of Shield 1 to the medium for stable expression of protein; however, given that Shield 1 is a rapamycin-analog compound, there is a question about its cytotoxicity. Also, from the viewpoint of the fact that a degradation time of approximately four hours is still required for inhibition of the expression, a system enabling more rapid degradation and elimination has been demanded in the research field of cell cycle and the like.

In view of the foregoing circumstances, the present inventors focused on plant-specific protein degradation induced by a plant hormone, auxin, and by introducing this degradation pathway into budding yeast, the present inventors developed a method of inhibiting the expression of a protein of interest, in which the protein of interest is degraded in such an extremely short time as 15 to 30 minutes by the addition of auxin to the medium (Patent Document 1).

### PRIOR ART DOCUMENT

### Patent Document

[Patent Document 1] JP-A-2008-187958

### Non Patent Document

[Non Patent Document 1] Gossen and Bujard, Proc. Natl. Acad. Sci. USA, 89, 5547 to 5551, 1992
[Non Patent Document 2] Gossen et al. Science, 268, 1766 to 1769, 1995
[Non Patent Document 3] Banazynski et al. Cell, 126, 995 to 1004, 2006

### SUMMARY OF THE INVENTION

### Problems to be Solved by the Invention

However, it was revealed that an expression vector using an Arabidopsis TIR1 gene and a plant Aux/IAA gene described in the aforementioned Patent Document 1 could induce degradation of protein in budding yeast but not in a mammalian cell culture system. Unless it can be used in a mammalian cell system, it cannot be applied to the study of the regulation of the expression of mammalian protein including humans.

Accordingly, the present invention aims to provide a system which can induce the degradation of a protein of interest in a mammalian cell system reliably and stably within a short time.

### Means for Solving the Problems

In view of the above, the present inventors studied the reason for preventing the aforementioned system described in Patent Document 1 from inducing protein degradation in a mammalian cell culture system. As a result, it was revealed that this expression vector was functional under temperature conditions of approximately 24° C., while it did not at around 37° C., which is the temperature used for mammalian cell culture. In light of this, they carried out a further study focusing on a TIR1 gene. As a result, they have found that when the used TIR1 gene is derived from cotton, which is a plant capable of growing even under high temperature conditions, it cannot function at 37° C. as in the case with an Arabidopsis TIR1, whereas, in contrast, when a rice TIR1 gene is employed, surprisingly, it functions efficiently even at 37° C. and can rapidly induce degradation of a protein of interest in a mammalian cell culture system.

They have also found that when an expression vector in which a rice TIR1 family gene is linked between a virus promoter and IRES and a chimeric gene expressing a protein of interest labeled with a plant Aux/IAA family protein is linked downstream thereof is used, totally unexpectedly, degradation of the protein of interest is induced efficiently and rapidly.

Accordingly, the present invention provides a mammalian cell inducible for protein degradation, the degradation of a protein of interest being induced by an auxin, wherein the mammalian cell has both a TIR1 family protein gene from rice and a chimeric gene expressing a protein of interest labeled with a plant Aux/IAA family protein.

The present invention also provides the aforementioned mammalian cell inducible for protein degradation, wherein the mammalian cell has an expression vector having a TIR1 family protein gene from rice linked between a virus promoter and IRES and the aforementioned chimeric gene linked downstream thereof transferred thereinto.

The present invention also provides a method for producing a mammalian cell inducible for protein degradation, the degradation of a protein of interest being induced by an auxin, wherein the method includes transferring a TIR1 family protein gene from rice and a chimeric gene expressing a protein of interest labeled with a plant Aux/IAA family protein into a host mammalian cell.

The present invention also provides the aforementioned method for producing a mammalian cell inducible for protein degradation, wherein the aforementioned gene transfer includes transferring an expression vector having a TIR1 family protein gene from rice linked between a virus promoter and IRES and the aforementioned chimeric gene linked downstream thereof.

The present invention also provides a gene transfer vector for producing a mammalian cell inducible for protein degradation, the degradation of a protein of interest being induced by an auxin, wherein the vector has a TIR1 family protein gene from rice and a chimeric gene expressing a protein of interest labeled with a plant Aux/IAA family protein.

The present invention also provides the aforementioned gene transfer vector, wherein the vector is an expression vector having a TIR1 family protein gene from rice linked between a virus promoter and IRES and the aforementioned chimeric gene linked downstream thereof.

The present invention also provides a method for inducing degradation of a protein of interest, including adding an auxin to the aforementioned mammalian cell inducible for protein degradation.

### Effects of the Invention

Since the use of the mammalian cell of the present invention allows reliable and specific induction of degradation of a protein of interest within 15 to 30 minutes by the addition of an auxin, it is useful as a tool for the functional analysis of a certain protein and in the field of drug development targeting the function of a certain protein, etc.

### BRIEF DESCRIPTION OF THE DRAWINGS

FIG. 1 shows the results of a thermostability experiment of the Arabidopsis TIR1, cotton TIR1, and rice TIR7 genes.

FIG. 2 is a conceptual diagram showing the production of the vector of the invention used for expressing rice TIR1 and a protein of interest in mammalian cells.

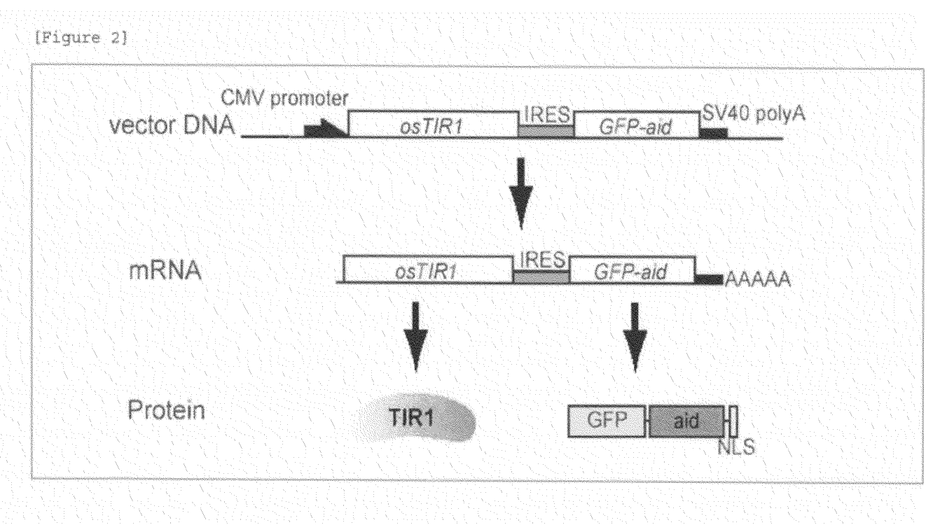

FIG. 3 shows induction of degradation of a protein of interest in monkey cells (COS1), Chinese hamster cells (CHO), and mouse cells (NIH3T3).

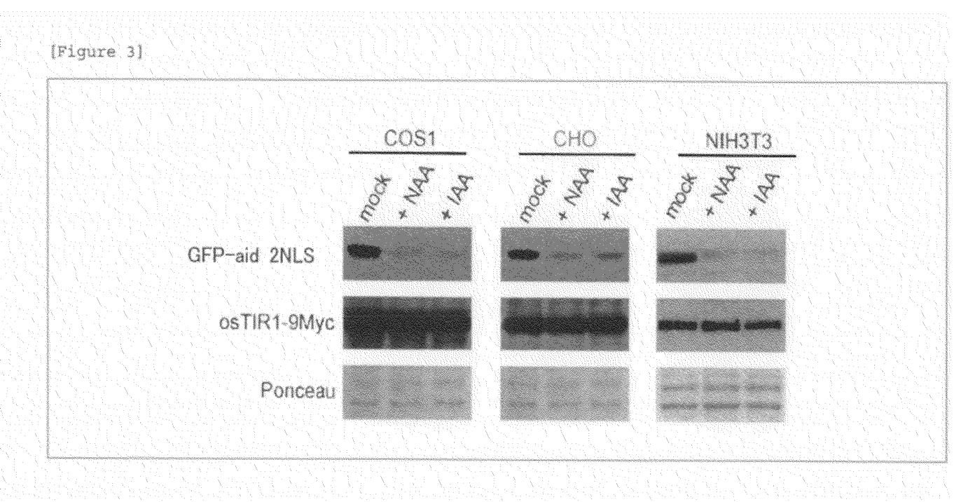

FIG. 4 shows a time course of induction of degradation of a protein of interest in human HEK293 cells.

FIG. 5 is a diagram showing the difference in the effect of induction of degradation of a labeled protein of interest between (A) the case in which an expression vector having a TIR1 family protein gene from rice linked between a virus promoter and IRES and a chimeric gene expressing a labeled protein of interest linked downstream thereof is transferred and (B) the case in which an expression vector having a chimeric gene expressing a labeled protein of interest linked between a virus promoter and IRES and a TIR1 family protein gene from rice linked downstream thereof is transferred.

### MODES FOR CARRYING OUT THE INVENTION

The mammalian cell inducible for protein degradation of the present invention contains (1) a TIR1 family protein gene from rice and (2) a chimeric gene expressing a protein of interest labeled with a plant Aux/IAA family protein. This combination of the TIR1 family protein gene and the chimeric gene expressing a protein of interest labeled with an Aux/IAA family protein is formed to establish a degradation pathway based on a plant ubiquitinating enzyme complex in the mammalian cell. That is, protein degradation by the ubiquitin-proteasome system is known in various eukaryotes such as animals, plants, fungi, and the like. In this degradation system, three enzymes, namely ubiquitin-activating enzyme (E1), ubiquitin-conjugating enzyme (E2), and ubiquitin-ligase (E3) act to bind ubiquitin to a target protein, and the resulting polyubiquitinated target protein is specifically recognized by proteasome and then degraded. As such a ubiquitin ligase, an E3 ubiquitinating enzyme complex (SCF complex) is reported. The SCF complex is composed of four subunits, namely F-box protein, Skp1 protein, Cullin-1 protein, and Rbx1 protein. The plant SCF complex contains TIR1 family protein as the F-box protein, and it was recently elucidated that this TIR1 family protein is the receptor for auxin, which is a growth hormone, and upon binding of auxin, it recognizes an Aux/IAA family protein, which is a repressor in the auxin signal transduction system, whereby degrading the protein. In this respect, the present inventors successfully made a degradation system of a protein of interest labeled with an Aux/IAA family protein function in mammalian cells, other than plants, by allowing a rice TIR1 family protein to recognize an Aux/IAA family protein, using auxin as an inducer.

The mammalian cell inducible for protein degradation of the present invention can degrade the expressed protein of interest at any time by adding an auxin. In other words, the mammalian cell inducible for protein degradation of the present invention expresses, for example, an SCF complex containing a rice TIR1 family protein and an Aux/IAA family protein-labeled protein of interest. Thus, in the co-existence of an auxin, the rice TIR1 family protein in the aforementioned SCF complex accepts an auxin, whereby the TIR1 family protein recognizes the Aux/IAA family protein and polyubiquitination of the Aux/IAA family protein is initiated. Thereafter, proteasome degrades the protein of interest labeled with the polyubiquitinated Aux/IAA family protein. Therefore, for example, the degradation of the expressed protein of interest can be regulated by either adding or not adding an auxin.

The TIR1 family protein gene used in the present invention is from rice. As described above, one derived from Arabidopsis, which is used in Patent Document 1, and one derived from cotton do not function at around 37° C., which is the growing temperature of the mammalian cell. Examples of the TIR1 family protein gene from rice include a TIR1 gene, an AFB1 gene, an AFB2 gene, an AFB3 gene, an FBX14 gene, and an AFB5 gene, among which a TIR1 gene is particularly preferable. More specifically, the gene registered in the National Center for Biotechnology Information (NCBI) under Accession No. NM_{—}001059194 (Gene ID: 4335696), Os04g0395600, or the gene registered in the above database under Accession No. EAY93933, OsI_{—}15707 is preferable.

The aforementioned TIR1 family gene from rice may be, for example, natural DNA extracted from rice or DNA synthesized by genetic engineering. Also, the aforementioned TIR1 family gene may be, for example, DNA containing exons and introns or cDNA composed of exons. The aforementioned TIR1 family gene may be, for example, full-length genomic DNA or full-length cDNA. Also, the aforementioned TIR1 family gene may be a partial sequence of genomic DNA or a partial sequence of cDNA as long as the expressed protein functions as a TIR1 family protein. In the present invention, to “function as a TIR1 family protein” means, for example, to recognize the Aux/IAA family protein in the presence of an auxin. This is because that, as described above, once a rice TIR1 family protein can recognize the Aux/IAA family protein, it can degrade an Aux/IAA family protein-labeled protein of interest. It is speculated that at this time a rice TIR1 family protein forms an SCF complex (an E3 ubiquitinating enzyme complex) with other subunits (Skp1 protein, Cullin protein, and Rbx1 protein) in the mammalian cell inducible for protein degradation of the present invention.

The mammalian cell inducible for protein degradation of the present invention preferably further contains a promoter sequence regulating the transcription of the aforementioned TIR1 family protein gene from rice. In this way, the rice TIR1 family protein can be more reliably expressed. No limitation is imposed on the aforementioned promoter, and for example, it can be appropriately determined according to the cell type and the like.

In the present invention, the aforementioned chimeric gene is a chimeric gene expressing a protein of interest labeled with an Aux/IAA family protein. The expressed protein of interest may be labeled with the aforementioned Aux/IAA family protein, and although no limitation is imposed on the form in which it exists, for example, it is preferably a fusion protein containing the protein of interest and the Aux/IAA family protein. The Aux/IAA family protein may be added to, for example, either the N-terminal side or the C-terminal side of the protein of interest.

Further, no limitation is imposed on the positional relationship between the gene of interest and the Aux/IAA family gene as long as both of these genes are functionally positioned such that the expressed protein of interest is labeled with the aforementioned Aux/IAA family protein. As a specific example, the aforementioned Aux/IAA family gene is preferably positioned adjacent to the aforementioned gene of interest upstream (5′ side) or downstream (3′ side) thereof. As long as the protein of interest is expressed and labeled with the Aux/IAA family protein, for example, the aforementioned Aux/IAA family gene may lie within the gene of interest.

No limitation is imposed on the kind of the aforementioned Aux/IAA family gene as long as it is a plant Aux/IAA family gene. Although no limitation is imposed on the kind of the aforementioned plant, an Arabidopsisan IAA17 gene is preferable. Specific examples of the Aux/IAA family gene include an IAA1 gene, an IAA2 gene, an IAA3 gene, an IAA4 gene, an IAA5 gene, an IAA6 gene, an IAA7 gene, an IAA8 gene, an IAA9 gene, an IAA10 gene, an IAA11 gene, an IAA12 gene, an IAA13 gene, an IAA14 gene, an IAA15 gene, an IAA16 gene, an IAA17 gene, an IAA18 gene, an IAA19 gene, an IAA20 gene, an IAA26 gene, an IAA27 gene, an IAA28 gene, an IAA29 gene, an IAA30 gene, an IAA31 gene, an IAA32 gene, an IAA33 gene, and IAA34 gene. The aforementioned mammalian cell inducible for protein degradation may contain any one or more than one of the aforementioned Aux/IAA family genes. For example, the sequence of the Arabidopsis Aux/IAA family gene is registered in The Arabidopsis Information Resource (TAIR), and Accession No. of each gene is shown below: IAA1 gene (AT4G14560), IAA2 gene (AT3G23030), IAA3 gene (AT1G04240), IAA4 gene (AT5G43700), IAA5 gene (AT1G15580), IAA6 gene (AT1G52830), IAA7 gene (AT3G23050), IAA8 gene (AT2G22670), IAA9 gene (AT5G65670), IAA10 gene (AT1G04100), IAA11 gene (AT4G28640), IAA12 gene (AT1G04550), IAA13 gene (AT2G33310), IAA14 gene (AT4G14550), IAA15 gene (AT1G80390), IAA16 gene (AT3G04730), IAA17 gene (AT1G04250), IAA18 gene (AT1G51950), IAA19 gene (AT3G15540), IAA20 gene (AT2G46990), IAA26 gene (AT3G16500), IAA27 gene (AT4G29080), IAA28 gene (AT5G25890), IAA29 gene (AT4G32280), IAA30 gene (AT3G62100), IAA31 gene (AT3G17600), IAA32 gene (AT2G01200), IAA33 gene (AT5G57420), and IAA34 gene (AT1G15050).

The aforementioned Aux/IAA family gene may be, for example, natural DNA or DNA synthesized by genetic engineering. Also, the aforementioned Aux/IAA family gene may be, for example, DNA containing exons and introns or cDNA composed of exons. The aforementioned Aux/IAA family gene may be, for example, full-length genomic DNA or full-length cDNA. Also, the aforementioned Aux/IAA family gene may be a partial sequence of genomic DNA or a partial sequence of cDNA as long as the expressed protein functions as the Aux/IAA family protein.

When the aforementioned Aux/IAA family gene is the aforementioned partial sequence, examples thereof include a DNA sequence of domain II of the Aux/IAA family protein. A specific example of the amino acid sequence of the aforementioned domain II is described in JP-A-2008-187958.

The mammalian cell inducible for protein degradation of the present invention preferably further contains a promoter sequence regulating the transcription of the aforementioned chimeric gene. In this way, the aforementioned Aux/IAA family protein-labeled protein of interest can be more reliably expressed. No limitation is imposed on the aforementioned promoter, and for example, it can be appropriately determined according to the cell type and the like.

In the present invention, the aforementioned gene of the protein of interest may be an endogenous gene present in the genome of the mammalian cell or an exogenous gene transferred into the mammalian cell. Also, for example, the aforementioned exogenous gene may be or may not be integrated into the genomic DNA. In the former case, for example, the exogenous gene is integrated in the genomic DNA by the use of a gene transfer vector to which the exogenous gene is linked. Also, in the latter case, for example, the aforementioned gene transfer vector and the like are present as plasmids, and the aforementioned gene of interest is preferably functionally linked to the origin of replication of the aforementioned gene transfer vector.

In the present invention, the mammalian cell preferably contains a Skp1 gene, a Cullin gene, and an Rbx1 gene. Each of these genes is preferably an endogenous gene of the mammalian cell. It is speculated that in the mammalian cell inducible for protein degradation of the present invention, an SCF complex is composed of proteins expressed by these genes (that is, Skp1 protein, Cullin protein, and Rbx1 protein) and the TIR1 family protein expressed by the TIR1 family protein gene from rice.

In the present invention, examples of the mammalian cell include cells obtained from humans, mice, rats, rabbits, and the like. Examples of the cell include culture cells such as HeLa cells, CHO cells, MCF, HEK293, HepG2, NIH3T3, COS cells, and DT40; primary culture cells; hematopoietic stem cells; hematopoietic cells and blood cells such as B cells, T cells, white blood cells, monocytes and macrophages, red blood cells, and platelets; fertilized oocytes; and ES cells. Further, other cells such as various tissue cells may also be used.

The method for producing a mammalian cell inducible for protein degradation of the present invention can be carried out, for example, by the steps of (1) transferring the TIR1 family gene from rice into a mammalian cell, which is a host, and (2) transferring the plant Aux/IAA family gene into the mammalian cell, which is the host, to form a chimeric gene expressing the aforementioned Aux/IAA family protein-labeled protein of interest.

In the present invention, no particular limitation is imposed on the order of the aforementioned steps (1) and (2), and these steps may be performed simultaneously. Transfer of the rice family gene in the aforementioned step (1) and transfer of the Aux/IAA family gene in the aforementioned step (2) may each be carried out, for example, using separate vectors; however, it is preferable to perform gene transfer using one vector having both the aforementioned rice family gene and Aux/IAA family gene inserted therein.

In the present invention, as described above, the gene of interest may be an endogenous gene present in the genomic DNA of the mammalian cell or an exogenous gene. When it is the aforementioned endogenous gene, for example, the Aux/IAA family gene is transferred into a mammalian cell, where it is functionally linked to the endogenous gene of interest to form a chimeric gene in the aforementioned step (2). Alternatively, it may also be possible to form a chimeric gene in which the Aux/IAA family gene is linked to the gene of interest and transfer the resulting chimeric gene into a mammalian cell to insert it into the genome by recombination with the genome.

When the gene of interest is an exogenous gene, for example, the exogenous gene may be transferred into the aforementioned mammalian cell before transferring the Aux/IAA family gene. Also, when the gene of interest is an exogenous gene, for example, it may be transferred into the mammalian cell with the Aux/IAA family gene. Specifically, a chimeric gene in which the Aux/IAA family gene and the gene of a protein of interest are functionally linked may be produced in advance, which may then be transferred into the mammalian cell.

A case in which the gene of interest is an exogenous gene and the TIR1 family gene and the chimeric gene are transferred by recombination using a vector will be explained.

Firstly, a rice TIR1 gene is integrated into a vector to produce a TIR1 gene transfer vector. No limitation is imposed on the kind of the vector, and for example, it can be appropriately determined according to the kind of the mammalian cell and the like. Specific examples thereof include a plasmid vector and a virus vector. Examples of the aforementioned plasmid vector include pCMV, pcDNA, and pACT, and examples of the aforementioned virus vector include the adenovirus expression system.

The TIR1 gene transfer vector preferably contains a promoter sequence regulating the transcription of the aforementioned TIR1 family gene. No limitation is imposed on the aforementioned promoter, and for example, it can be appropriately determined according to the kind of the mammalian cell and the like. Specific examples thereof include a CMV promoter, an SV40 promoter, and a GAL4-binding sequence. Normally, the aforementioned promoter is preferably functionally linked upstream (5′ side) of the TIR1 family gene. Further, it may be a cell species-specific promoter and an organ-specific promoter. Also, a TIR1 family gene lacking a promoter sequence may be, for example, integrated downstream of an endogenous promoter of the host cell or the host animal. In that case, the TIR1 family gene may be, for example, targeted at a specific gene or integrated randomly.

Also, the TIR1 gene transfer vector may further contain a selection marker coding sequence since it allows confirmation of whether or not the transfer of the vector into the mammalian cell has been successful. No limitation is imposed on the aforementioned selection marker coding sequence, and examples thereof include a sequence encoding a marker such as a known drug resistance marker, a fluorescent protein marker, and a cell surface receptor marker. No limitation is imposed on the aforementioned drug resistance marker, and examples thereof include a neomycin resistance marker, a puromycin resistance marker, and a hygromycin resistance marker. Examples of the aforementioned fluorescent protein marker include Green Fluorescent Protein (GFP) and Enhanced GFP (EGFP). Also, examples of an enzyme marker include luciferase and β-galactosidase. These selection marker coding sequences may be synthesized by PCR and the like according to their sequences or prepared from commercial vectors having the aforementioned selection marker coding sequences. It is to be noted that when the TIR1 gene transfer vector is used in combination with a chimeric gene transfer vector to be described later, the selection makers for the aforementioned TIR1 gene transfer vector and the chimeric gene transfer vector are preferably different. Further, the TIR1 family gene may be, for example, functionally linked to other protein genes or tag sequences and expressed as a protein containing the aforementioned protein and the TIR1 protein (such as a fusion protein) or a tag-labeled TIR1 protein.

Meanwhile, the gene of interest and the plant Aux/IAA family gene are linked to a vector to produce a chimeric gene transfer vector. As described above, the chimeric gene is a gene expressing an Aux/IAA family protein-labeled protein of interest. In the aforementioned vector, no limitation is imposed on the positions of the aforementioned gene of interest and the Aux/IAA family gene as long as they are related in such a way that the labeled protein can be expressed, as described above. As a specific example, the aforementioned Aux/IAA family gene is preferably positioned adjacent to the aforementioned gene of interest upstream (5′ side) or downstream (3′ side) thereof, and also, the Aux/IAA family gene may lie within the gene of interest.

No limitation is imposed on the aforementioned vector, and examples thereof include the same ones as those exemplified above. Also, the chimeric gene transfer vector preferably contains a promoter sequence regulating the transcription of the aforementioned chimeric gene. Examples of the aforementioned promoter include the same ones as those exemplified above. Further, the chimeric gene transfer vector may further contain a selection marker coding sequence such as those exemplified above since it allows confirmation of whether or not the transfer of the vector into the mammalian cell has been successful.

Subsequently, the aforementioned TIR1 gene transfer vector and the chimeric gene transfer vector are transferred into the mammalian cell, which is a host cell (step (1) and step (2)). No limitation is imposed on the order of transfer of the aforementioned two vectors.

No particular limitation is imposed on the method for transferring a vector, and for example, it can be appropriately determined according to the kind of the vector, the type of the host cell, and the like. Examples of the transfer method include calcium phosphate transfection, DEAE dextran transfection, and electroporation, and in addition, a method using a retrovirus vector, an adenovirus vector, and the like.

In the present invention, it is efficient to use an expression vector having both the TIR1 gene and the chimeric gene inserted therein. Specifically, it is preferable to use a vector having a virus promoter and IRES (a ribosome binding site in mRNA) into which the above genes are inserted. Normally, such a pIRES vector is designed to be used by having the gene of interest to be expressed inserted between the virus promoter and IRES. However, the present inventors have found that degradation of the protein of interest is induced efficiently and rapidly by producing an expression vector having the TIR1 family gene from rice linked between a virus promoter and IRES and the aforementioned chimeric gene linked downstream thereof (refer to Example 2 to be described later). Examples of the promoter of this expression vector include a CMV promoter and an SV40 promoter, of which a CMV promoter is preferable.

Also, when the gene of interest is present in the genomic DNA, for example when the gene of interest is an endogenous gene of the mammalian cell or when the gene of interest is an exogenous gene but already integrated into the genomic DNA of the mammalian cell, the expression vector can be produced as follows. In that case, an Aux/IAA family gene transfer vector, which has the Aux/IAA family gene inserted therein, is used instead of a chimeric gene transfer vector. This gene transfer vector is preferably constructed, for example, so as to allow the Aux/IAA family gene to be integrated into such a site in the genomic DNA of the mammalian cell that enables labeling of the protein of interest with the Aux/IAA family protein when the protein is expressed. That is, the aforementioned gene transfer vector may be constructed such that the gene of interest in the genomic DNA and the Aux/IAA family gene are functionally linked to form a chimeric gene by recombination with the genomic DNA. Integration of Aux/IAA family in the above manner enables the expression of an Aux/IAA family-labeled protein of interest. It is to be noted that the chimeric gene transfer vector can be constructed by those skilled in the art based on, for example, the locus and the sequence of the gene of interest in the genomic DNA.

Degradation of the protein of interest is induced by allowing an auxin to act on the mammalian cell inducible for protein degradation of the present invention. The degradation of the protein of interest is reliable and extremely rapid. For example, it is almost completely degraded within 15 to 30 minutes.

For the induction of degradation of the protein of interest, no limitation is imposed on the amount of auxin to be added. For example, it can be appropriately determined according to the kind of the auxin. As a specific example, 1 μM to 1 mM auxin is added, and preferably 20 μM to 500 μM auxin is added to the medium.

Examples of the auxin include 1-naphthaleneacetic acid (NAA) and indole-3-acetic acid. Also, additional examples include a group of compounds having similar physiological activities to the aforementioned NAA and the like, such as 2,4-dichlorophenoxyacetic acid, 4-chlorophenoxyacetic acid, (2,4,5-trichlorophenoxy)acetic acid, 1-naphthaleneacetamide, 2,4-dichlorophenoxyacetic acid, and 4-para-chloroacetic acid. For example, a precursor which acquires a physiological activity of auxin by metabolism can also be used. For example, a substance which is converted into a substance having an auxin activity by esterase and β-oxidation enzyme in a host cell is preferable. Specific examples thereof include indole-3-acetic acid methyl ester and indole-3-butyric acid.

The auxin may be added to a medium containing the aforementioned cell.

Since the addition of the auxin rapidly induce degradation of the protein of interest as described above, the effect of the protein of interest can be studied by comparing with the case in which auxin is not added. More specifically, studies can be conducted by a method including adding an auxin to the aforementioned cell to induce degradation of the expressed protein of interest, and subsequently removing the aforementioned auxin to inhibit degradation of the newly expressed protein of interest; and a method including adding an auxin to the aforementioned cell to induce degradation of the expressed protein of interest, and subsequently adding an auxin-inhibiting substance to inhibit degradation of the newly expressed protein of interest.

### EXAMPLES

Examples of the present invention will be described hereinbelow. However, the present invention is not limited by the following Examples.

### Reference Example 1

### 1. Production of a Budding Yeast Strain

EGFP was determined as the protein to be degraded, and a budding yeast strain which degrades EGFP by the addition of NAA was produced. It is to be noted the promoter sequence and the gene sequence of the budding yeast used are registered in the SGD web site, which is http://www.yeastgenome.org/.

### (1) Arabidopsis TIR1-Expressing Yeast Strain (YNK2)

After cleaving a pRS306-GAL plasmid vector with SalI and XhoI, the resulting fragment was allowed to self-ligate. The pRS306-GAL plasmid vector is disclosed in a document (L. Drury, G. Perkins and J. Diffley “The Cdc4/34/53 pathway targets Cdc6p for proteolysis in budding yeast” EMBO J 16, 5966 to 5976, 1997). By this process, the SalI site in the multicloning site of the aforementioned plasmid vector was eliminated. The Arabidopsis TIR1 gene (TAIR Accession No. AT1G04250.1) (1785 bp) was amplified by PCR using the following primer set 1, and the amplified product thus obtained was cloned into the SpeI-NotI site in the aforementioned plasmid vector. The vector thus obtained is referred to as pMK26.

<Primer set 1>
F primer 7
(SEQ ID NO: 1)
5′-AGCTAGACTAGTATGCAGAAGCGAATAGCCTT-3′
R primer 8
(SEQ ID NO: 2)
5′-ATCGATGCGGCCGCAGATCTGCTAGTCGACTAATCCGTTAGT
AGTAATGA-3′

A SalI-BglII fragment was cleaved out of a pYM18 plasmid vector to prepare a 9Myc-tag segment (435 bp) and the segment was cloned into the SalI-BglII site of the aforementioned vector pMK26. The pYM18 plasmid vector is disclosed in a document (C. Janke, M. Magiera, N. Rathfelder, C. Taxis, S. Reber, H. Maekawa, A. Moreno-Borchart, G. Doenges, E. Schwob, E. Schiebel, and M. Knop. “A versatile toolbox for PCR-based tagging of yeast genes: new fluorescent proteins, more markers and promoter substitution cassettes.” Yeast 21, 974 to 962, 2004). The vector thus obtained is referred to as pMK27. This vector pMK27 was cleaved at the StuI site in the URA3 marker and the resulting vector was used for transformation of a budding yeast wild strain W303-1a, thereby the aforementioned plasmid vector was inserted into the URA3 site of the budding yeast genome by homologous recombination. The recombinant thus obtained was referred to as an Arabidopsis TIR1-expressing strain “YNK2”. It is to be noted that, in YNK2, a vector-derived GAL1-10 promoter is positioned upstream of the TIR1 gene.

The genotypes of the wild strain W303-1a and YNK2 are shown below.

### W303-1a

MATa ade2-1 ura3-1 his3-11, 15 trp1-1 leu2-3, 112 can1-100

### YNK2

MATa ade2-1 his3-11, 15 trp1-1 leu2-3, 112 can1-100 ura3-1: URA3-GAL1-10 promoter-Arabidopsis TIR1 (pMK27 integrated)

### Production Method of a Cotton TIR1-Expressing Yeast Strain

A cotton TIR1 gene (NCBI, Accession No. DQ659621) was amplified from the cotton cDNA library by PCR using the following primer set. The Arabidopsis TIR1 gene was removed from pMK26 by treatment with SpeI and SalI, into which the amplified product was cloned.

F primer
(SEQ ID NO: 3)
5′-GGGACTAGTATGCATAAGAAAATGGCGTTTTCG-3
R primer
(SEQ ID NO: 4)
5′-CCCGTCGACTGAAAGCCTCAATCCAGAATCTTC-3′

Using the same technique as that used for transfer of Arabidopsis TIR1, the cotton TIR1 was transferred into budding yeast.

### <Genotype of a Cotton TIR1-Expressing Yeast Strain>

MATa ade2-1 his3-11, 15 trp1-1 leu2-3, 112 can1-100 ura3-1: URA3-GAL1-10 promoter-cotton TIR1

### Production Method of a Rice TIR1-Expressing Yeast Strain

A rice TIR1 gene (NCBI, Accession No. NM_{—}001059194) was amplified from the rice cDNA library by PCR using the following primer set. The Arabidopsis TIR1 gene was removed from pMK26 by treatment with SpeI and SalI, into which the amplified product was cloned.

F primer
(SEQ ID NO: 5)
5′-GGGGATCCATGACGTACTTCCCGGAGGAGGT-3
R primer
(SEQ ID NO: 6)
5′-CCCGTCGACTAGGATTTTAACAAAATTTGGTG-3′

Using the same technique as that used for transfer of Arabidopsis TIR1, the rice TIR1 was transferred into budding yeast.

### <Genotype of a Rice TIR1-Expressing Yeast Strain>

MATa ade2-1 his3-11, 15 trp1-1 leu2-3, 112 can1-100 ura3-1: URA3-GAL1-10 promoter-rice TIR1

### Reference Example 2

### 1. Production of a Budding Yeast Strain

(1) An auxin Degron Strain for an Endogenous Protein Mcm4 (mcm4-ad)

An endogenous protein Mcm4 was determined as the protein to be degraded, and a budding yeast strain which degrades Mcm4 by the addition of NAA was produced. The endogenous protein Mcm4 is a protein involved in DNA replication.

Firstly, in order to add a 5xGA linker (5xGly-Ala) to the coding region of IAA17, the coding region of IAA17 (746 bp) was amplified by PCR using IAA17 as a template and the following primer set 7. Since the R primer of the following primer set 7 contains a 5xGA linker, a 5xGA linker is added to the C′ terminus of the resulting amplified product. It is to be noted that the underlined sequence is the linker in the following SEQ ID NO: 8.

<Primer set 6>
F primer 142
(SEQ ID NO: 7)
5′-ACGTATGAATTCTGTATATGAGATAGTTGATTGTATGC-3′
R primer 172
(SEQ ID NO: 8)
5′-ATACGTGGTACCGAGCTCTGGCACCCGCTCCAGCGCCTGCACCAG
CTCCCAAGTCCTTAGATTCAATTTGAAGTTCTTCCTCGAGAGCTCTGC
TCTTGCACTTCTC-3′

The fragment to which the above linker was attached was cloned into the EcoRI-KpnI site of a pKL187 plasmid vector (A. Sanchez-Diaz, M. Kanemaki, V. Marchesi and K. Labib “Rapid depletion of budding yeast proteins by fusion to a heat-inducible degron.” Sci STKE 223, PL8, 2004) to produce a plasmid vector for transferring the coding sequence of IAA17 by 1-step PCR. The resulting vector is referred to as pMK38. This vector pMK38 was amplified by PCR using the following primer set 8. Subsequently, the TIR1-expressing YNK2 strain obtained in the aforementioned (1) was directly transformed with the amplified product thus obtained, and through homologous recombination, CUP1 promoter-IAA17 was transferred into the 5′ part of MCM4 in the budding yeast genome. The recombinant thus obtained is referred to as “YNK4”. It should be noted that insertion into the genome was confirmed by PCR.

<Primer set 7>
F primer 180
(SEQ ID NO: 9)
5′-TTCTTTAAGAACATCTTCAATACTAAATAAGACAACCCATCTTCA
GTTATATTAAGGCGCGCCAGATCTG-3′
R primer 181
(SEQ ID NO: 10)
5′-GAGCTGGAGTTATTATCCTCTTTTGTTGGAGAGCTAGACTGTTGA
GACATGGCACCCGCTCCAGCGCCTG-3′

The genotype of YNK4 is shown below.

### YNK4

MATa ade2-1 his3-11, 15 trp1-1 leu2-3, 112 can1-100 ura3-1: URA3-GAL1-10 promoter-Arabidopsis TIR1 (pMK27 integrated)
mcm4: CUP1 promoter-mcm4-ad (kanMX)

Budding yeast strains each having one of the cotton TIR1 gene and the rice TIR1 gene transferred therein instead of the aforementioned Arabidopsis-derived TIR1 gene were produced.

### 2. Growth Experiment of a Budding Yeast Strain

The YNK4 strain was spotted on a YPDCu agar medium without NAA added (2% peptone, 1% powdered yeast, 2% glucose, 0.1 mM CuSO₄, and 2% agar) and a YPGNAA agar medium with NAA added (2% peptone, 1% powdered yeast, 2% galactose, 0.1 mM NAA, and 2% agar) at a predetermined number of cells per dish (5×10⁵, 5×10⁴, 5×10³, 5×10², and 5×10 cells), followed by culturing at 24° C. for two days. Thereafter, the growth of the cells was observed. As a control, the growth of a budding yeast wild strain W303-1a and the YNK2 strain produced in Reference Example 1 was also observed in a similar manner.

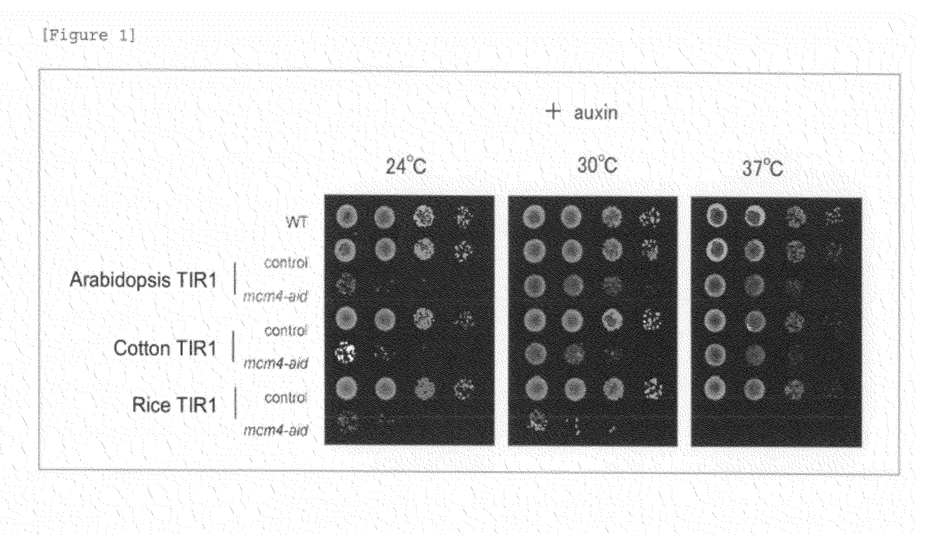

The results are shown in FIG. 1. This Figure shows the results of the culture at 24° C., 30° C., and 37° C., and in the Figure, with respect to each strain, the numbers of cells spotted are 5×10⁵, 5×10⁴, 5×10³, 5×10², and 5×10 cells, respectively, from left.

From these results, it was found that when the Arabidopsis TIR1 gene was used, while it induced degradation of the protein of interest at 24° C., it did not induce degradation well at 30° C. or higher. Meanwhile, given that both cotton and rice can grow under a relatively high temperature, they are assumed to have excellent thermostability; however, it was found that when the cotton TIR1 gene was used, it could not induce degradation of the protein of interest well at 30° C. or higher.

In contrast, it was revealed that when the rice TIR1 gene was used, it was able to induce degradation of the protein of interest well at 30° C. and 37° C.

### Example 1

### Production of a Mammalian Cell Strain

### (1) Production of an Expression Vector and Transformation

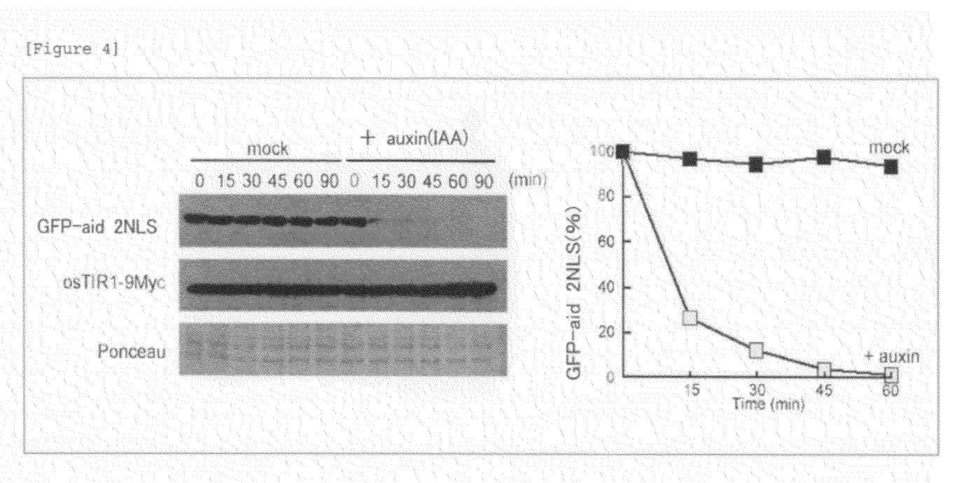

The EGFP segment of pIRES2-AcGFP1 (Clontech) was removed by treatment with restriction enzymes BstXI and NotI, into which EGFP-IAA17, which was amplified using pMK42 (an EGFP-IAA17-containing plasmid produced by the inventors) as a template and the following primer set, was inserted (the amplified sequence is shown in SEQ ID NO: 13). By this process, EGFP-IAA17 was cloned downstream of IRES. The resulting plasmid was treated with restriction enzymes XhoI and SmaI, and into this site, rice TIR1 tagged with 9Myc at its C terminus was inserted. By this process, the rice TIR1 was inserted between a CMV promoter and IRES, and the resulting plasmid was referred to as pNHK60. The pNHK60 was transferred into the proliferating COS1, CHO, and NIH3T3 cells using Lipofectamine 2000 (Invitrogen) for transient expression. The HEK293 cell shown in FIG. 4 is a HEK293 strain stably expressing pNHK60, which was obtained by transfer of the pNHK60 under the aforementioned conditions and subsequent selection by the addition of G418 to a medium.

<Primer set 8>
F-primer
(SEQ ID NO: 11)
5′-CAGTGAATTCCCACAACCATGGTGAGCAAGGGCGAGGA-3′
R-primer
(SEQ ID NO: 12)
5′-GGTCATGCGGCCGCTGGGTACCTTAAACCTTACG-3′

### (2) Induction of Degradation of a Protein of Interest in a Mammalian Cell Strain

In the experiment of transient expression, the cells were cultured in a medium to which 500 μM IAA or NAA was added, and after five hours, the cells were collected, from which extracts were prepared. In the experiment using the stably-expressing strain shown in FIG. 4, after addition of 500 μM IAA, the cells were collected at times indicated in the Figure and extracts were prepared. The factors were detected by Western blotting.

### (3) Western Blot

The protein thus extracted was separated by SDS-PAGE and then transferred to a nitrocellulose membrane. Using an anti-Myc antibody (mouse monoclonal 9E10) and an anti-GFP antibody (mouse monoclonal 9E1), each kind of protein was detected by Western blotting. It is to be noted that an HRP-labeled IgG antibody was used as the secondary antibody. For migration control, total extracted proteins were detected by Ponceau staining. Since the Arabidopsis TIR1 gene is tagged with 9Myc, the TIR1 protein can be detected by Western blotting with the aforementioned Myc antibody. Also, EGFP, which is a protein to be degraded in the complex protein, can be detected by Western blotting with the GFP antibody.

### Example 2

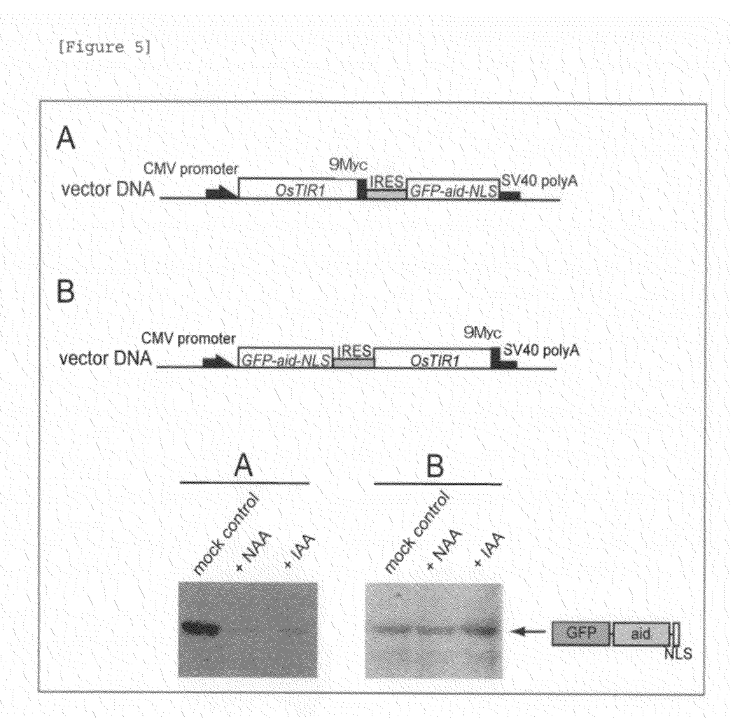

In a similar manner to Example 1, the induction of degradation of a labeled protein of interest was compared between the case in which an expression vector having the TIR1 family protein gene from rice linked between a virus promoter and IRES and the chimeric gene expressing a labeled protein of interest linked downstream thereof was transferred (corresponding to A in FIG. 5) and the case in which an expression vector having the chimeric gene expressing a labeled protein of interest linked between a virus promoter and IRES and the TIR1 family protein gene from rice linked downstream thereof was transferred (corresponding to B in FIG. 5). Specifically, monkey-derived COS1 cells were transiently transfected with plasmids each containing a DNA construct of either A or B in FIG. 5, and after one day, cultured in a medium containing auxin (500 μM NAA or IAA) for 5 hours. After extraction of a protein sample, the expressed GFP-aid-NLS marker protein was detected by Western blotting using the GFP antibody. As a result, when DNA construct A was used, the marker protein was found to be efficiently depleted, whereas when DNA construct B was used, no outstanding depletion was observed (FIG. 5).

Normally, a pIRES vector is designed to be used by having a gene of interest to be expressed inserted between a virus promoter and IRES. The results given above completely defy the common knowledge about the pIRES vector.

1. A mammalian cell inducible for protein degradation, the degradation of a protein of interest being induced by an auxin, wherein the mammalian cell has both a TIR1 family protein gene from rice and a chimeric gene expressing a protein of interest labeled with a plant Aux/IAA family protein.

2. The mammalian cell inducible for protein degradation according to claim 1, wherein the mammalian cell has an expression vector having the TIR1 family protein gene from rice linked between a virus promoter and IRES and the chimeric gene linked downstream thereof transferred thereinto.

3. A method for producing a mammalian cell inducible for protein degradation, the degradation of a protein of interest being induced by an auxin, wherein the method comprises transferring a TIR1 family protein gene from rice and a chimeric gene expressing a protein of interest labeled with a plant Aux/IAA family protein into a host mammalian cell.

4. The method for producing a mammalian cell inducible for protein degradation according to claim 3, wherein the transfer of the genes comprises transferring an expression vector having the TIR1 family protein gene from rice linked between a virus promoter and IRES and the chimeric gene linked downstream thereof.

5. A gene transfer vector for producing a mammalian cell inducible for protein degradation, the degradation of a protein of interest being induced by an auxin, wherein the vector has a TIR1 family protein gene from rice and a chimeric gene expressing a protein of interest labeled with a plant Aux/IAA family protein.

6. The gene transfer vector according to claim 5, wherein the vector is an expression vector having the TIR1 family protein gene from rice linked between a virus promoter and IRES and the chimeric gene linked downstream thereof.

7. A method for inducing degradation of a protein of interest, comprising adding an auxin to the cell according to claim 1 or 2.


SEQUENCE LISTING (14 sequences)

SEQ ID NO: 1 - DNA, 32 bp, Artificial Sequence
  Description of Artificial Sequence Synthetic designed DNA based on Arabidopsis thaliana TIR1 gene
agctagactagtatgcagaagcgaatagcctt

SEQ ID NO: 2 - DNA, 50 bp, Artificial Sequence
  Description of Artificial Sequence Synthetic designed DNA based on Arabidopsis thaliana TIR1 gene
atcgatgcggccgcagatctgctagtcgactaatccgttagtagtaatga

SEQ ID NO: 3 - DNA, 33 bp, Artificial Sequence
  Description of Artificial Sequence Synthetic designed DNA based on Gossypium spp. gene
gggactagtatgcataagaaaatggcgttttcg

SEQ ID NO: 4 - DNA, 33 bp, Artificial Sequence
  Description of Artificial Sequence Synthetic designed DNA based on Gossypium spp. gene
cccgtcgactgaaagcctcaatccagaatcttc

SEQ ID NO: 5 - DNA, 31 bp, Artificial Sequence
  Description of Artificial Sequence Synthetic designed DNA based on Oryza sativa gene
ggggatccatgacgtacttcccggaggaggt

SEQ ID NO: 6 - DNA, 32 bp, Artificial Sequence
  Description of Artificial Sequence Synthetic designed DNA based on Oryza sativa gene
cccgtcgactaggattttaacaaaatttggtg

SEQ ID NO: 7 - DNA, 38 bp, Artificial Sequence
  Description of Artificial Sequence Synthetic designed DNA based on Arabidopsis thaliana IAA17 gene
acgtatgaattctgtatatgagatagttgattgtatgc

SEQ ID NO: 8 - DNA, 106 bp, Artificial Sequence
  Description of Artificial Sequence Synthetic designed DNA based on Arabidopsis thaliana IAA17 gene
atacgtggtaccgagctctggcacccgctccagcgcctgcaccagctcccaagtccttagattcaatttgaagttcttcctcgagagctctgctcttgcacttctc

SEQ ID NO: 9 - DNA, 70 bp, Artificial Sequence
  Description of Artificial Sequence Synthetic designed DNA based on PMK38
ttctttaagaacatcttcaatactaaataagacaacccatcttcagttatattaaggcgcgccagatctg

SEQ ID NO: 10 - DNA, 70 bp, Artificial Sequence
  Description of Artificial Sequence Synthetic designed DNA based on pMK38
gagctggagttattatcctcttttgttggagagctagactgttgagacatggcacccgctccagcgcctg

SEQ ID NO: 11 - DNA, 38 bp, Artificial Sequence
  Description of Artificial Sequence Synthetic designed DNA based on pMK42 gene
cagtgaattcccacaaccatggtgagcaagggcgagga

SEQ ID NO: 12 - DNA, 34 bp, Artificial Sequence
  Description of Artificial Sequence Synthetic designed DNA based on pMK42 gene
ggtcatgcggccgctgggtaccttaaaccttacg

SEQ ID NO: 13 - DNA, 1513 bp, Artificial Sequence
  Description of Artificial Sequence Synthetic designed DNA based on EGEP-IAA17 gene
ccacaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggagctggtgcaggcgctggagcgggtgccgggcccatgatgggcagtgtcgagctgaatctgagggagactgagctgtgtcttggtcttcccggtggagatacagtggctccggtaaccggaaacaagagagggttctcagagacggttgatctgaagctaaatctgaataatgagcctgcaaacaaggaaggatctacgactcatgacgtcgtgacttttgattccaaggagaagagtgcttgtcctaaagatccagccaaacctccggccaaggcacaagttgtgggatggccaccggtgagatcataccggaagaacgtgatggtttcctgccaaaaatcaagcggtggcccggaggcggcggcgttcgtgaaggtatcaatggacggagcaccgtacttgaggaaaatcgatttgaggatgtataaaagctacgatgagctttctaatgctttgtccaacatgttcagctcttttaccatgggcaaacatggaggagaagaaggaatgatagacttcatgaatgagaggaaattgatggatttggtgaatagctgggactatgttccctcttatgaagacaaagacggtgattggatgctcgtcggcgacgttccttggccaatgttcgtcgatacatgcaagcgtttacgtctcatgaaaggatcggatgccattggtctcgctccgagggcgatggagaagtgcaagagcagagctggtgctggcgcccccaagaagaaaagaaaggtccctaaaaagaaacgtaaggtttaagcggccgc

SEQ ID NO: 14 - protein, 6 aa, Artificial Sequence
  Description of Artificial Sequence Synthetic 5xGly-Ala linker
GGGGGA